(19) United States
(12) Patent Application Publication
(10) Pub. No.: US 2009/0324754 A1
(43) Pub. Date: Dec. 31, 2009

(54) EUCALYPTUS EXTRACT, METHOD OF PREPARATION AND THERAPEUTIC USES THEREOF

(75) Inventors: Christel Fiorini-Puybaret, Toulouse (FR); Bernard Fabre, Belberaud (FR); Cécile Chauvin, Grenoble (FR); Philippe Joulia, Villenouvelle (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Pierre Fabre Medicament, Boulogne (FR)

(21) Appl. No.: 12/309,754

(22) PCT Filed: Jul. 27, 2007
(86) PCT No.: PCT/FR07/01309

§ 371 Date: Jan. 28, 2009

(30) Foreign Application Priority Data
Aug. 1, 2006 (FR) 0607201

(51) Int. Cl. A61K 36/61; A61P 25/00; A61K 31/11

(52) U.S. Cl. 424/742; 514/700

(57) ABSTRACT

The present invention relates to the use of a Eucalyptus for preparation of a drug or a dietary supplement intended for treatment and/or prevention of affections or pathologies caused by neuromediator recapture disorder. The present invention also relates to a new enriched extract of Eucalyptus characterized in that it contains at least one compound having the formula (I) or any one of its diastereoisomers in which R1 forms a C═CH2 substituent with the carbon to which it is joined, a formula (II) substituent and R2 which represents an isobutyl, α-isobutyl or β-isobutyl substituent; and the method of preparation thereof.

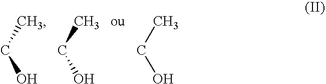

The present invention relates to the use of a Eucalyptus extract in the preparation of a medicament or of a food supplement for the treatment and/or prevention of diseases or pathologies arising from a disorder of the reuptake of the following neuromediators: dopamine, serotonin and/or noradrenaline. Said extract is preferably enriched with at least one of the compounds of formula (I) or any one of the diastereoisomeric forms thereof:

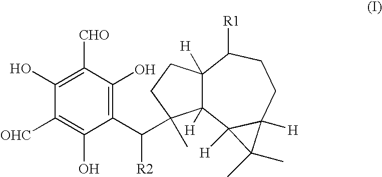

in which R1, together with the carbon atom to which it is

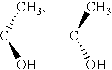

bonded, forms a C═CH2 group or a group or

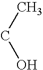

and R2 represents an isobutyl, α-isobutyl or β-isobutyl group.

The present invention relates also to the use of at least one of the compounds of formula (I) above in the preparation of a medicament or of a food supplement for the treatment and/or prevention of diseases or pathologies arising from a disorder of the reuptake of said neuromediators.

There are many species of Eucalyptus (more than 600), most of which are native to Australia and Tasmania, a small number to New Guinea and East Malaysia. The Eucalyptus belong to the myrtle family, and the origin of their name lies in the characteristic shape of their flowers. The word Eucalyptus in fact means “well covered”, alluding to the operculum (formed of fused petals) which covers the stamen of the flowers. Eucalyptus are generally beautiful, large trees which can reach a height of 80 to 100 m in their country of origin (Australia) or 3 to 20 m in more temperate climates. Their bark, which is smooth, comes away in long strips of pale to greyish colour. They are generally characterised by foliar dimorphism. In the case of Eucalyptus globulus Labill, the juvenile leaves are oval-oblong, glaucous green, circled with blue, embracing and sessile, while the adult leaves are falciform, greyish-green and pendant, have a twisted petiole and are oriented vertically (equivalence of the two faces). The flower buds are formed of a calyx of quadrangular pyramid shape, covered by an operculum which lifts during flowering to reveal numerous stamen with long white filaments and yellow anthers. The fruits are capsules having a diameter of from 2 to 2.5 cm which contain black or brownish seeds.

In phytotherapy, three species are principally used in the European pharmacopoeia: Eucalyptus globulus Labill, E. polybractea R. T. Baker and Eucalyptus smithii R. T. Baker. The leaves of the oldest branches (which leaves are falciform and petiolate), the essential oil and the eucalyptol obtained therefrom are used.

Eucalyptus leaves are conventionally used by the oral and local routes to treat diseases of the respiratory system (bronchitis, inflammation of the throat, blocked nose, cold, etc.) or by application to treat wounds, skin ulcers, etc.

The essential oil of Eucalyptus and eucalyptol (or 1,8-cineol) are used in numerous preparations for treating respiratory tract diseases owing to their antiseptic, mucolytic and expectorant activities. The essential oil is used as a repellent and in veterinary medicine.

The pharmacological properties of the essential oil of Eucalyptus known to date are: antimicrobial, expectorant and anti-tussive properties (WICHTL M. and ANTON R., 1999—Plantes thèrapeutiques—177-179), anti-inflammatory and anti-asthmatic properties (JUERGENS et al., 2003—Anti-inflammatory activity of 1,8-cineol (eucalyptol) in bronchial asthma: a double-bind placebo controlled trial—Respir. Med., 97 (3), 250-256), anti-diabetic properties (SWANSTON et al., 1990—Traditional plant treatments for diabetes. Studies in normal and streptozotocin diabetic rats—Diabetologia, 33 (8), 462-464), anti-histaminic properties (IKAWATI Z. et al., 2001—Screening of several Indonesian medicinal plants for their inhibitory effect on histamine release from RBL-2H3cells—J. Ethnopharmacol., 75 (2-3), 248-256), anti-cancer properties (TAKASAKI et al., 2000—Cancer chemopreventive activity of euglobal-G1 from leaves of Eucalyptus grandis—Cancer Lett., 155 (1) 61-65), anti-viral properties (TAKASAKI et al., 1990—Structures of euglobal-G1, -G2 and G3 from Eucalyptus grandis, three new inhibitors of Epstein virus activation—Chem. Pharm. Bull. 38 (5), 1444-1446) and anti-HIV properties (WICHTL M. and ANTON R., 1999—Plantes thèrapeutiques—177-179).

Unexpectedly and surprisingly, the Applicants have revealed the use of a Eucalyptus extract in the preparation of a medicament or of a food supplement for the treatment and/or prevention of diseases or pathologies arising from a disorder of neuromediator reuptake.

The field of the present invention is, therefore, a Eucalyptus extract for which valuable pharmacological properties have been observed and new therapeutic uses have accordingly been envisaged. The present invention does not relate to the essential oil of Eucalyptus as such, for which an abundant bibliography has been noted.

The medicament or food supplement is advantageously intended for the treatment and/or prevention of pathologies arising from a disorder of neuromediator reuptake selected from the group:
neurological illnesses, diseases or disorders,
psychiatric illnesses, diseases or disorders,
memory, attention and vigilance disorders related to neurological and psychiatric illnesses, diseases or disorders,
functional somatic disorders,
dependence on addictive substances.

The treatment and/or prevention of said diseases or pathologies preferably comprises inhibiting the reuptake of the neuromediators.

Within the scope of the present invention, “neuromediators” are understood as being: dopamine and/or serotonin and/or noradrenaline.

Within the scope of the present invention, “Eucalyptus” is understood as meaning the species belonging preferably to the subgenera Eudesmia, Symphomyrtus and Corymbia and more especially the following species: Eucalyptus globulus L., Eucalyptus pulverulenta Sims, Eucalyptus kartzoffiana L. A. S. Johnson 1 Blaxell, Eucalyptus macrocarpa Hook., Eucalyptus cinerea F. Muell.ex Benth., Eucalyptus dorrigoensis (Blakely) L. A. S. Johnson 1 K. D. Hill, Eucalyptus leptopoda Benth., Eucalyptus occidentalis Endl., Eucalyptus viridis R. T. Baker, Eucalyptus polybractea R. T. Baker and Eucalyptus smithii R. T. Baker.

Those examples illustrate the present invention without, however, limiting the scope thereof.

The Eucalyptus extract is advantageously obtained from Eucalyptus leaves, flowers, fruits, stems or trunk, preferably from Eucalyptus leaves.

The Eucalyptus extract used according to the present invention is advantageously characterised in that it comprises at least one compound of formula (I) or any one of the diastereoisomeric forms thereof.

Said formula (I) includes inter alia four compounds of the macrocarpal family, namely:
macrocarpal A: (5-((1R)-1-((11S,7R)-7-hydroxy-3,3,7,11-tetramethyltricyclo(6.3.0.0(2,4))undec-11-yl)-3-methylbutyl)-2,4,6-trihydroxybenzene-1,3-dicarbaldehyde) in which R1, together with the carbon atom to which it is bonded, forms the group

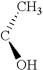

and R2 represents a β-isobutyl group.

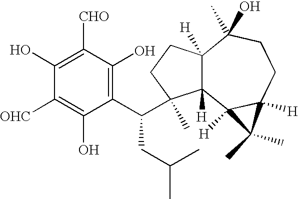

Empirical formula: C₂₈H₄₀O₆

Molecular weight: 472 g/mol

The fraction by weight of macrocarpal A in the Eucalyptus extract according to the present invention is advantageously greater than or equal to 0.1% and strictly less than 3%.
macrocarpal B: (5-((1S)-1-((11S,7R)-7-hydroxy-3,3,7,11-tetramethyltricyclo(6.3.0.0(2,4))undec-11-yl)-3-methyl-butyl)-2,4,6-trihydroxybenzene-1,3-dicarbaldehyde) in which R1, together with the carbon atom to which it is bonded, forms the group

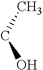

and R2 represents an α-isobutyl group.

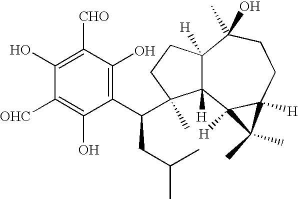

Empirical formula: C₂₈H₄₀O₆

Molecular weight: 472 g/mol

The fraction by weight of macrocarpal B in the Eucalyptus extract according to the present invention is advantageously greater than or equal to 0.1% and strictly less than 3%.
macrocarpal C: (5-((1R)-1-((11S)-3,3,11-trimethyl-7-methylenetricyclo(6.3.0.0(2,4))undec-11-yl)-3-methyl-butyl)-2,4,6-trihydroxybenzene-1,3-dicarbaldehyde) in which R1, together with the carbon atom to which it is bonded, forms the C═CH2 group and R2 represents a β-isobutyl group.

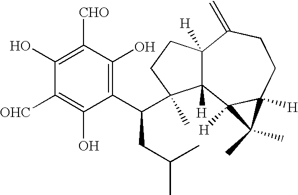

Empirical formula: C₂₈H₃₈O₅

Molecular weight: 454 g/mol

The fraction by weight of macrocarpal C in the Eucalyptus extract according to the present invention is advantageously greater than or equal to 0.1% and strictly less than 3%.
macrocarpal G : (5-(1-(3,3,11-trimethyl-7-methylene-tricyclo(6.3.0.0(2,4))undec-11-yl)-3-methylbutyl)-2,4,6-trihydroxybenzene-1,3-dicarbaldehyde) in which R1, together with the carbon atom to which it is bonded, forms a C═CH2 group and R2 represents an isobutyl group

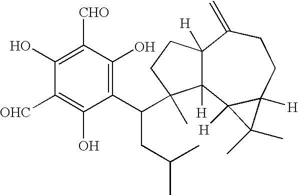

Empirical formula: C₂₈H₃₈O₅

Molecular weight: 454 g/mol

The fraction by weight of macrocarpal G in the Eucalyptus extract according to the present invention is advantageously greater than or equal to 0.1% and strictly less than 5%.

Said Eucalyptus extract is obtained by an extraction process carried out starting from conventional steps known to the person skilled in the art.

The Eucalyptus (Eucalyptus sp.) leaves, flowers, fruits, stems or trunk, or a mixture of those parts, are ground and then extracted with an organic solvent which can be an alkane (pentane, hexane, heptane, octane, cyclohexane), an ether oxide (tetrahydrofuran, dioxane, diethyl ether), an ester (ethyl acetate, isopropyl acetate), an alcohol (methanol, ethanol, propanol, isopropanol, butanol, octanol), a ketone (methyl ethyl ketone, methyl isobutyl ketone), a halogenated hydrocarbon (chloroform, dichloro-methane) or a mixture of water and water-miscible organic solvent(s) (for example an aqueous-alcoholic mixture).

The extraction is carried out in a plant/solvent ratio of from approximately 1/1 to approximately 1/20 and can be repeated 2 to 3 times. The temperature of the extraction solvent can be equal to or higher than ambient temperature and can reach the boiling point of the solvent used. The time for which the plant is in contact with the solvent is from approximately 30 minutes to approximately 72 hours.

A solid/liquid separation is then carried out, the plant being separated from the solvent by filtration or centrifugation.

The resulting filtrate can be either:
evaporated to dryness directly by total evaporation of the extraction solvent, and constitutes the final extract;
or concentrated to a greater or lesser degree. In the case of a mixed extraction solvent (for example an aqueous-alcoholic mixture), the concentration is continued until the organic solvent present has evaporated off. In the case of an organic solvent, an amount of water is added to the resulting concentrate. A liquid-liquid purification step is carried out by adding to the aqueous phase an immiscible solvent, which can be an alkane (for example hexane), an ether oxide (for example diethyl ether), an ester (for example ethyl acetate), an alcohol (for example butanol), a ketone (for example methyl ethyl ketone) or a halogenated hydrocarbon (for example chloroform). One, two or three liquid-liquid extractions are carried out. The combined organic phases can be dried over sodium sulphate before being evaporated to dryness.

The solutions obtained are concentrated in vacuo and at a temperature from ambient temperature to the boiling point.

Drying of the final extract is carried out by lyophilisation or by more conventional drying means known to the person skilled in the art (nebulisation, oven, etc.). The drying temperatures preferably do not exceed about 60° C.

The extract can be stabilised by addition of an antioxidant such as, for example, ascorbic acid or citric acid, in amounts of from approximately 0.05 to approximately 1 g per 100 g of dry extract.

A wholly remarkable aspect of the present invention is that the pharmacological properties of the Eucalyptus extract of inhibiting the reuptake of the neuromediators are all the more interesting, the more said extract is enriched with at least one of the compounds of formula (I) or any one of the diastereoisomeric forms thereof.

Accordingly, said Eucalyptus extract is preferably enriched with at least one compound of formula (I).

Within the scope of the present invention, “Eucalyptus extract enriched with macrocarpal A” is understood as meaning a Eucalyptus extract in which the fraction by weight of macrocarpal A is greater than or equal to 3% and strictly less than 90%, preferably greater than or equal to 3% and less than 50%, more preferably greater than or equal to 3% and less than 40% and yet more preferably greater than or equal to 3% and less than 20%.

Within the scope of the present invention, “Eucalyptus extract enriched with macrocarpal B” is understood as meaning a Eucalyptus extract in which the fraction by weight of macrocarpal B is greater than or equal to 3% and strictly less than 90%, preferably greater than or equal to 3% and less than 50%, more preferably greater than or equal to 3% and less than 40% and yet more preferably greater than or equal to 3% and less than 20%.

Within the scope of the present invention, “Eucalyptus extract enriched with macrocarpal C” is understood as meaning a Eucalyptus extract in which the fraction by weight of macrocarpal C is greater than or equal to 3% and strictly less than 90%, preferably greater than or equal to 3% and less than 50%, more preferably greater than or equal to 3% and less than 40% and yet more preferably greater than or equal to 3% and less than 20%.

Within the scope of the present invention, “Eucalyptus extract enriched with macrocarpal G” is understood as meaning a Eucalyptus extract in which the fraction by weight of macrocarpal G is greater than or equal to 5% and strictly less than 90%, preferably greater than or equal to 5% and less than 50%, more preferably greater than or equal to 5% and less than 40% and yet more preferably greater than or equal to 5% and less than 20%.

The Applicants have demonstrated the effect of a Eucalyptus extract on dopamine and/or noradrenaline and/or serotonin reuptake.

Owing to its pharmacological properties of inhibiting the reuptake of those neuromediators, said extract is useful especially for the preparation of a medicament or of a food supplement for the treatment and/or prevention of numerous diseases or pathologies resulting from a dopamine and/or serotonin and/or noradrenaline deficiency.

Among the diseases or pathologies which can be treated and/or prevented by means of a Eucalyptus extract according to the present invention, the following may be mentioned by way of non-limiting examples:
neurological illnesses, diseases or disorders:
such as neurodegenerative diseases (Alzheimer's disease, Huntington's chorea, Parkinson's disease, cerebral vascular accidents, cranial traumatism), amyotrophic lateral sclerosis, senile dementia, fronto-temporal dementia, vascular dementia, migraine, neuropathic pain of central origin;
psychiatric illnesses, diseases or disorders:
such as depression (endogenous, resistant, reactive or iatrogenic depression), breakdown, schizophrenia, bipolar syndrome, general anxiety, stress-related diseases, panic attacks, obsessive compulsive disorders, post-traumatic stress syndromes, attention and hyperactivity disorders, eating disorders (especially bulimia, anorexia), phobia (especially agoraphobia), autism;
memory, attention and vigilance disorders related to neurological and psychiatric illnesses, diseases or disorders;
functional somatic disorders:
such as chronic fatigue syndrome, fibromyalgia, irritable bowel syndrome, gastro-oesophageal reflux, loss of libido, erectile dysfunction, urinary incontinence;
dependence on addictive substances:
especially nicotine, alcohol, opiates, cannabinoids, psychostimulants.

In fact, the medicament or food supplement according to the invention is advantageously intended to induce withdrawal from nicotine, alcohol, opiates, cannabinoids or psychostimulants and to prevent relapse in abstinent persons.

The medicament or food supplement according to the present invention can therefore advantageously be used as a replacement treatment for addictive substances, and for preventing or treating withdrawal-related depressive syndrome.

The person skilled in the art will be able to recognise other pathologies whose treatment requires such inhibition.

The Applicants mention here, in a non-limiting manner, a number of bibliographic references which mention the link between the pathologies and their treatment by means of a triple dopamine and/or serotonin and/or noradrenaline reuptake inhibitor. An example of each “group” has been given.

Dopamine, serotonin and noradrenaline cooperate in the development and survival of neurons (Lauder J. M., Trends Neurosci, 1993, 16; 233). Some neurological pathologies such as Parkinson's disease (Hornykiewicz O., Adv Cytopharmacol. 1971, 1; 369) are the result of a dopamine deficiency; monoamine oxidase inhibitors, which increase dopamine, serotonin and noradrenaline levels, are used to treat Parkinson's disease and other neurological diseases (Ebadi M., Curr Drug Targets. 2006, 7; 1513). The Eucalyptus extract according to the present invention can therefore advantageously be used in the treatment of such neurological diseases.

Depression is a frequent mood pathology, characterised by feelings of intense sadness, pessimistic thoughts, self-depreciation, often accompanied by a loss of drive, enthusiasm and libido. The inability to feel pleasure in normally pleasurable experiences, which is also known by the name anhedonia, is also regarded as a frequent symptom in depression. At present, depression is treated with selective serotonin reuptake inhibitors, such as fluoxetine, citalopram or paroxetine, selective noradrenaline reuptake inhibitors, such as reboxetine, or by mixed serotonin and noradrenaline reuptake inhibitors, such as milnacipran or venlafaxine. However, an important role in pleasure and motivation has been attributed to the dopamine neurons projecting to a region of the brain called the nucleus accumbens (Koob G. F. Sem. Neurosci. 1992, 4, 139; Salamone J. D. Behav. Brain Res. 1994, 61, 117). The symptoms of depression can therefore advantageously be treated by a dopamine, serotonin and noradrenaline reuptake inhibitor such as the Eucalyptus extract according to the present invention.

The absorption of addictive substances, including nicotine, raises the extracellular dopamine levels in the ventral striatum in animals (Di Chiara G and Imperato A., Proc Natl Acad Sci USA. 1988, 85; 5274) and in humans (Brody et al., Am J Psychiatry, 2004, 161; 1211). Tobacco withdrawal can be accompanied by a depressive syndrome (Wilhelm K et al., Drug Alcohol Rev, 2006, 25; 97). The Eucalyptus extract according to the present invention can therefore advantageously be used as a replacement treatment for addictive substances, such as nicotine, and for preventing or treating withdrawal-related depressive syndrome.

Functional disorders, also called somatotropic disorders, are disorders which relate to the major physiological functions and which would be due not to organic lesions but to the manner in which organs (liver, heart, etc.) function. Functional somatic disorders can be at the origin of a disease which will manifest itself later. Among such disorders, fibromyalgia is a disorder which combines diffuse and localised pain, chronic fatigue, depressive symptoms, and memory and concentration disorders (Rooks D S., Curr Opin Rheumatol. 2007, 19; 111). The symptoms of fibromyalgia are treated by mixed noradrenaline/serotonin reuptake inhibitors (Vitton O., Hum Psychopharmacol. 2004, 19 Suppl 1:S27). The addition of a component favouring dopaminergic tonicity, as in the Eucalyptus extract according to the present invention, can therefore advantageously be used as a medicament or food supplement for the treatment and/or prevention of functional somatic disorders.

Said medicament is advantageously in an oral or injectable form.

The oral form is advantageously selected from the group consisting of tablet, gelatin capsule, capsule, liquid preparations such as syrups, drinkable solutions or powders for drinkable suspensions.

Said food (or nutraceutical or dietetic) supplement is advantageously packaged in unit dose form, namely in forms of presentation such as gelatin capsules, lozenges, tablets, pills and other similar forms, as well as powder sachets, liquid ampoules, bottles provided with a drop counter, and the other analogous forms of liquid or powder preparations that are to be taken in measured doses of small amounts.

The results obtained from a Eucalyptus extract enriched with at least one compound of formula (I) or any one of the diastereoisomeric forms thereof according to the present invention show that the benefits of the present invention can be extended to any composition based on at least one compound of formula (I) or any one of the diastereoisomeric forms thereof, whether the latter is obtained by chemical means, biochemical means or from a plant extract.

The present invention therefore relates also to the use of at least one compound of formula (I) or any one of the diastereoisomeric forms thereof in the preparation of a medicament or of a food supplement for the treatment and/or prevention of neurological or psychiatric diseases or pathologies and related disorders, of functional somatic syndromes and of dependence on addictive substances, arising from a disorder of neuromediator reuptake. The treatment and/or prevention of said diseases or pathologies preferably comprises inhibiting neuromediator reuptake.

Owing to their pharmacological properties of inhibiting the reuptake of those neuromediators, said compounds of formula (I) and their diastereoisomeric forms thereof are useful especially for the preparation of a medicament or of a food supplement for the treatment and/or prevention of numerous diseases or pathologies resulting from a dopamine and/or serotonin and/or noradrenaline deficiency.

Among the diseases or pathologies which can be treated and/or prevented by means of said compounds according to the present invention, the following may be mentioned by way of non-limiting examples:
neurological illnesses, diseases or disorders:
such as neurodegenerative diseases (Alzheimer's disease, Huntington's chorea, Parkinson's disease, cerebral vascular accidents, cranial traumatism), amyotrophic lateral sclerosis, senile dementia, fronto-temporal dementia, vascular dementia, migraine, neuropathic pain of central origin;
psychiatric illnesses, diseases or disorders:
such as depression (endogenous, resistant, reactive or iatrogenic depression), breakdown, schizophrenia, bipolar syndrome, general anxiety, stress-related diseases, panic attacks, obsessive compulsive disorders, post-traumatic stress syndromes, attention and hyperactivity disorders, eating disorders (especially bulimia, anorexia), phobia (especially agoraphobia), autism;
memory, attention and vigilance disorders related to neurological and psychiatric illnesses, diseases or disorders;
functional somatic disorders:
such as chronic fatigue syndrome, fibromyalgia, irritable bowel syndrome, gastro-oesophageal reflux, loss of libido, erectile dysfunction, urinary incontinence;
dependence on addictive substances:
especially nicotine, alcohol, opiates, cannabinoids, psychostimulants.

In fact, the medicament or food supplement according to the invention is advantageously intended to induce withdrawal from nicotine, alcohol, opiates, cannabinoids or psychostimulants and to prevent relapse in abstinent persons.

The medicament or food supplement according to the present invention can therefore advantageously be used as a replacement treatment for addictive substances, and for preventing or treating withdrawal-related depressive syndrome.

The person skilled in the art will be able to recognise other pathologies whose treatment requires such inhibition.

The present invention relates advantageously to the use of macrocarpal A, macrocarpal B, macrocarpal C and/or macrocarpal G in the preparation of a medicament or food supplement for the treatment and/or prevention of neurological or psychiatric diseases or pathologies and related disorders, of functional somatic syndromes and of dependence on addictive substances, arising from a dopamine and/or serotonine and/or noradrenaline reuptake disorder.

Tests carried out with the compounds of formula (I) and their diastereoisomeric forms thereof have shown that those compounds act on the inhibition of dopamine and/or serotonin and/or noradrenaline reuptake.

Said medicament is advantageously in an oral or injectable form.

The compounds of formula (I) and their diastereoisomeric forms according to the present invention can be obtained by purification of a plant extract or by chemical or biochemical synthesis as described in Total Synthesis of (−)-Macrocarpal C. Stereoselective Coupling Reaction with a Novel Hexasubstituted Benzene Cr(CO)₃ Complex as a Biomimetic Chiral Benzyl Cation Equivalent/Tanaka, Tetsuaki; Mikamiyama, Hidenori; Maeda, Kimiya; Iwata, Chuzo; In, Yasuko; Ishida, Toshimasa/Journal of Organic Chemistry (1998), 63(26), 9782-9793.

Said compounds can be isolated from “the eucalyptus extract” or from “the extract enriched with at least one macrocarpal of formula (I)”. Techniques permitting its purification are chromatographic techniques that are conventional for the person skilled in the art. The extracts are fractionated on a preparative column having a reverse phase, preferably Symetry Shield®, 5 μm (Waters), as the stationary phase and an acetonitrile/-water/trifluoroacetic acid mixture in the proportions 95/5/0.1% as the mobile phase.

The purity of such a fraction in respect of compound of formula (I) is greater than or equal to 90%.

The macrocarpal A, macrocarpal B, macrocarpal C and/or macrocarpal G purity of such a fraction is preferably greater than or equal to 90%.

Within the scope of the present invention, it is possible reasonably to envisage that the macrocarpal A and/or macrocarpal B and/or macrocarpal C and/or macrocarpal G can be used in the preparation of a medicament or food supplement for the treatment and/or prevention of obesity and overweight.

The present invention relates also to an enriched Eucalyptus extract, characterised in that it comprises at least one compound of formula (I) or any one of the diastereoisomeric forms thereof:

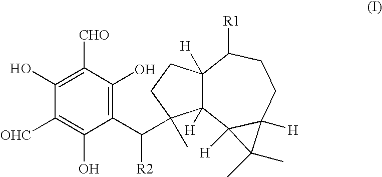

in which R1, together with the carbon atom to which it is bonded, forms a C═CH2 group or a group

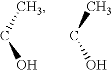

or

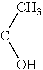

and R2 represents an isobutyl, α-isobutyl or β-isobutyl group and in that:
the fraction by weight of macrocarpal A is greater than or equal to 3% and strictly less than 90%, preferably greater than or equal to 3% and less than 50%, more preferably greater than or equal to 3% and less than 40% and yet more preferably greater than or equal to 3% and less than 20%;
the fraction by weight of macrocarpal B is greater than or equal to 3% and strictly less than 90%, preferably greater than or equal to 3% and less than 50%, more preferably greater than or equal to 3% and less than 40% and yet more preferably greater than or equal to 3% and less than 20%;
the fraction by weight of macrocarpal C is greater than or equal to 3% and strictly less than 90%, preferably greater than or equal to 3% and less than 50%, more preferably greater than or equal to 3% and less than 40% and yet more preferably greater than or equal to 3% and less than 20%;
and the fraction by weight of macrocarpal G is greater than or equal to 5% and strictly less than 90%, preferably greater than or equal to 5% and less than 50%, more preferably greater than or equal to 5% and less than 40% and yet more preferably greater than or equal to 3% and less than 20%.

The present invention therefore relates preferably to the use of said enriched Eucalyptus extract as a medicament or food supplement.

Finally, the present invention relates to a process for the preparation of such an enriched Eucalyptus extract.

The process for obtaining said extract comprises the following steps:
grinding Eucalyptus leaves and/or flowers and/or fruits and/or stems and/or trunk,
extracting at least once with an organic solvent or a mixture of water and water-miscible organic solvent(s).
The extraction is carried out in a plant/solvent ratio of from approximately 1/1 to approximately 1/20 and can be repeated 2 to 3 times. The temperature of the extraction solvent can be equal to or higher than ambient temperature and can reach the boiling point of the solvent used. The time for which the plant is in contact with the solvent is from approximately 30 minutes to approximately 72 hours.
The solvent is preferably selected from the group comprising an alkane (pentane, hexane, heptane, octane, cyclohexane), an ether oxide (tetrahydrofuran, dioxane, diethyl ether), an ester (ethyl acetate, isopropyl acetate), an alcohol (methanol, ethanol, propanol, isopropanol, butanol, octanol), a ketone (methyl ethyl ketone, methyl isobutyl ketone), a halogenated hydrocarbon (chloroform, dichloromethane) or a mixture of water and water-miscible organic solvents (for example an aqueous-alcoholic mixture).
The extraction solvent is preferably dichloromethane or isopropyl acetate.
In the case of a water-miscible extraction solvent, the filtrate is evaporated to dryness and then dissolved in a water-immiscible solvent.
In the case of a water-immiscible solvent, the filtrate is concentrated.
solid/liquid separation by techniques known to the person skilled in the art.

In a preferred embodiment of the invention, one or more liquid-liquid extractions are carried out by addition of a base, preferably sodium carbonate (Na₂CO₃). The combined basic aqueous phases are acidified by addition of acid, preferably hydrochloric acid (HCl), and then extracted by one to several liquid-liquid extractions carried out with a water-immiscible solvent. The acidification advantageously results in a pH of approximately 1.

The combined organic phases can be dried over sodium sulphate and then concentrated in vacuo at a temperature varying from ambient temperature to boiling point.

The concentrate is dried by conventional drying means (nebulisation, oven, etc.) at temperatures preferably not exceeding 60° C., and constitutes the extract enriched with macrocarpal G. The extract can be stabilised by addition of an antioxidant such as, for example, ascorbic acid or citric acid in amounts of from 0.05 to 1 g per 100 g of dry extract.

In a particular embodiment of the invention, the Eucalyptus extract or the so-called Eucalyptus extract “enriched with at least one compound of formula (I) or any one of the diastereoisomeric forms thereof” is likewise obtained by an extraction process using a supercritical fluid as the extraction solvent.

The Eucalyptus (Eucalyptus sp.) leaves, flowers, fruits, stems or trunk, or a mixture of those parts, are or are not ground and are then extracted with a supercritical fluid which can be carbon dioxide.

A first extraction using advantageously supercritical CO2 is carried out in the following way:
the temperature of the fluid is preferably from approximately 40° C. to approximately 80° C.; more preferably from approximately 40° C. to approximately 60° C.;
its pressure is preferably from approximately 80 bar to approximately 250 bar; more preferably from approximately 100 bar to approximately 200 bar
the time for which the extraction is carried out is preferably from approximately 1 hour to approximately 6 hours;
its flow rate will be adjust by the person skilled in the art according to the quantity of plant which has to be extracted and to the autoclave size. The fluid flow rate is preferably from approximately 2 to approximately 15 kg/hour; more preferably from 8 to approximately 12 kg/hour;
   for a quantity of plant which is from approximately 200 grams to approximately 1000 grams; which is preferably approximately 500 grams;
   for an autoclave size which is from approximately 2 litres to approximately 10 liters; which is preferably approximately 5 litres.

During that first extraction step, it is possible to add an organic co-solvent from the family of the alcohols (including ethanol), the ether oxides, the esters or a mixture of two or more of those solvents.

The plant so extracted can then optionally be subjected to a second extraction. The extraction liquid is advantageously supercritical CO2 with or without a co-solvent. The operating conditions are as follows:
the temperature of the fluid is preferably from approximately 40° C. to approximately 80° C.; more preferably from approximately 40° C. to approximately 60° C.;
its pressure is preferably from approximately 80 bar to approximately 250 bar; more preferably from approximately 100 bar to approximately 200 bar
The fluid flow rate is preferably from approximately 2 to approximately 15 kg/hour; more preferably from 8 to approximately 12 kg/hour;
   for a quantity of plant which is from approximately 200 grams to approximately 1000 grams; which is preferably approximately 500 grams;
   for an autoclave size which is from approximately 2 litres to approximately 10 liters; which is preferably approximately 5 litres.

The extraction is preferably carried out in a plant/co-solvent ratio by weight of from approximately 1/0.1 to 1/5.

The second extraction step can be repeated if necessary. The time for which the said extraction is carried out is preferably from approximately 1 hour to approximately 3 hours for each supplementary extraction step.

Evaporation of the resulting extract is then carried out.

The person skilled in the art will adapt the operating conditions of that process using supercritical liquid in order to obtain a Eucalyptus extract that is enriched to a greater or lesser degree.

Drying of the final extract is carried out by lyophilisation or by more conventional drying means known to the person skilled in the art (nebulisation, oven, etc.). The drying temperatures preferably do not exceed about 60° C.

The extract can be stabilised by addition of an antioxidant such as, for example, ascorbic acid or citric acid, in amounts of from approximately 0.05 to approximately 1 g per 100 g of dry extract.

The invention will be better understood with the aid of the following examples, which do not, however, limit the scope thereof.

### EXAMPLE 1

Preparation of a Eucalyptus globulus Extract

Eucalyptus globulus leaves are ground and then extracted with 5 volumes of ethanol at ambient temperature. The time for which the plant is in contact with the solvent is 48 hours.

The plant is separated from the solvent by filtration.

The filtrate obtained is dried in vacuo at a temperature of 60° C.

The extract so obtained will be used for the in vitro tests presented in Example 5.

The extract obtained comprises approximately:
0.65 g of macrocarpal A
0.7 g of macrocarpal B
0.4 g of macrocarpal C
1 g of macrocarpal G

per 100 g of dry extract.

### EXAMPLE 2

### Preparation of a Eucalyptus globulus Extract

Eucalyptus globulus leaves are ground and then extracted three times at reflux with 5 volumes of 50% v/v ethanol. The time for which the plant is in contact with the solvent is approximately one hour.

The plant is separated from the solvent by filtration.

The filtrate obtained is concentrated to 0.5 volume. Liquid-liquid purification is carried out by adding dichloromethane. Three liquid-liquid extractions are carried out. The organic phases are combined and dried over sodium sulphate. Drying of the final extract is carried out at 60° C. in vacuo.

The extract obtained comprises approximately:
1.3 g of macrocarpal A
1.4 g of macrocarpal B
0.8 g of macrocarpal C
2 g of macrocarpal G

per 100 g of dry extract.

### EXAMPLE 3

### Preparation of a Eucalyptus globulus Extract

Eucalyptus globulus leaves are ground and then extracted twice at reflux with 5 volumes of a 50% v/v ethanol/water mixture for approximately one hour.

The plant is separated from the solvent by filtration.

The filtrate obtained is concentrated and then stabilised by addition of citric acid in an amount of 0.1 g per 100 g of dry extract.

The concentrate is frozen and then dried by lyophilisation.

The extract obtained comprises approximately:
0.3 g of macrocarpal A
0.35 g of macrocarpal B
0.2 g of macrocarpal C
0.5 g of macrocarpal G

per 100 g of dry extract.

### EXAMPLE 4

537 g of Eucalyptus leaves are ground and then placed in an autoclave. They are extracted for 4 hours with supercritical CO2 at 40° C., 150 bar, with a flow rate of 10 kg/h.

The leaves so extracted are subjected to a second extraction using a supercritical CO2/ethanol mixture at 150 bar, 50° C. 1 volume of ethanol is used per 1 part by weight of plant. Extraction is carried out in that manner for 2 hours 15 minutes, and then the leaves are dried by passage of CO2on its own under the same operating conditions for 30 minutes.

273.7 g of extract are recovered and are dried by evaporating off the solvent. 27.9 g of an extract comprising 6.85 g of macrocarpal G per 100 g of dry extract are obtained in that manner.

### EXAMPLE 5

### Evaluation of the Extract of Eucalyptus globulus Leaves on Serotonin, Dopamine and Noradrenaline Reuptake

The uptake tests were carried out in vitro on synapses of rats.

1) Evaluation of Serotonin (or 5-HT) Reuptake

The protocol used for this evaluation is that described in Perovic, S. and Muller W. E. G., 1995—Pharmacological profile of hypericum extract: effect on serotonin uptake by postsynaptic receptors, Arzneim-Forsch. Drug Res., 45: 1145-1148.

The principle is as follows:

Synapses obtained from rat brains are incubated for 15 minutes at 37° C. with 0.1 μCi of [³H]-serotonin in the presence or absence (control) of the Eucalyptus globulus extract prepared according to Example 1 or of imipramine (reference) in a buffer comprising 118 mM NaCl, 5 mM KCl, 2.5 mM MgSO₄, 1.2 mM NaH₂PO₄, 25 mM NaHCO₃, 11 mM glucose, 10 μM EGTA and 50 μM ascorbic acid (pH=959 7.4). The baseline activity is determined by incubating the same mixture for 15 minutes at 37° C. in the presence of 10 μM imipramine in order to block the reuptake.

Following the incubation, the samples are filtered rapidly in vacuo through glass-fibre filters (GB/B, Packard) and rinsed twice with ice-cold incubation buffer in order to eliminate free [³H]-serotonin. The filters are dried and the retained radioactivity is measured by means of a scintillation counter (Topcount, Packard) using a scintillation cocktail (Microscint O, Packard).

The results are expressed as a percent inhibition of the control uptake of [³H]-serotonin (see Table 1)

2) Evaluation of Dopamine (or DA) Reuptake

The protocol used for this evaluation is that described in Janowsky A., Berger P., Vocci F., Labarca R., Skolnick P., and Paul S. M., 1996—Characterization of sodium-dependent [³H]GBR-12935 binding in brain: a radioligand for selective labelling of the dopamine transport complex, J. Neurochem., 46, 1272-1276.

The principle is as follows:

Synaptic medium (synapses of rat striatum) is incubated for 15 minutes at 37° C. with 0.1 μCi of [³H]-DA in the presence or absence (control) of the Eucalyptus globulus extract prepared according to Example 1 or of GBR 12909 (reference) in the buffer solution (see serotonin reuptake).

The baseline activity is determined by incubating the same mixture for 15 minutes at 37° C. in the presence of 10 μM GBR 12909 in order to block the reuptake.

Following the incubation, the samples are filtered rapidly in vacuo through glass-fibre filters (GB/B, Packard) and rinsed twice with ice-cold incubation buffer in order to eliminate free [³H]-dopamine. The filters are dried and the retained radioactivity is measured by means of a scintillation counter (Topcount, Packard) using a scintillation cocktail (Microscint O, Packard).

The results are expressed as a percent inhibition of the control uptake of [³H]-dopamine (see Table 1).

3) Evaluation of Noradrenaline (or NE) Reuptake

The protocol used for this evaluation is that described in Perovic, S. and Muller W. E. G., 1995—Pharmacological profile of hypericum extract: effect on serotonin uptake by postsynaptic receptors, Arzneim-Forsch. Drug Res., 45: 1145-1148.

The principle is as follows:

Synaptic medium (synapses of rat hypothalamus) is incubated for 20 minutes at 37° C. with 0.1 μCi of [³H]-NE in the presence or absence (control) of the Eucalyptus globulus extract prepared according to Example 1 or of protriptyline (reference) in the buffer solution (see serotonin reuptake).

The baseline activity is determined by incubating the same mixture for 20 minutes at 37° C. in the presence of 10 μM protriptyline in order to block the reuptake.

Following the incubation, the samples are filtered rapidly in vacuo through glass-fibre filters (GB/B, Packard) and rinsed twice with ice-cold incubation buffer in order to eliminate free [³H]-NE. The filters are dried and the retained radioactivity is measured by means of a scintillation counter (Topcount, Packard) using a scintillation cocktail (Microscint O, Packard).

The results are expressed as a percent inhibition of the control uptake of [³H]-noradrenaline (see Table 1).

4) Results:

The results are expressed as the percentage inhibition of reuptake of the neuromediator evaluated (see Table 1).

TABLE 1
Evaluation of the extract of Eucalyptus globulus leaves
on serotonin, dopamine and noradrenaline reuptake

| | Concentrations | |
|---|---|---|
| | (μg of dry extract/ | % |
| Test | ml of solution) | inhibition |
| Serotonin reuptake | 1 | 3 |
| | 10 | 43 |
| | 100 | 103 |
| Dopamine reuptake | 1 | 4 |
| | 10 | 91 |
| | 100 | 102 |
| Noradrenaline reuptake | 1 | −14 |
| | 10 | 11 |
| | 100 | 101 |

Significant inhibition of serotonin and dopamine reuptake is observed at 10 μg/ml, and significant inhibition of the reuptake of the three neurotransmitters is observed at 100 μg/ml.

### EXAMPLE 6

### Evaluation of a Eucalyptus Extract Enriched with Macrocarpal G vs Eucalyptus Extract without Macrocarpal G and vs Pure Macrocarpal G

Eucalyptus globulus leaves are ground and then extracted with 5 volumes of dichloromethane. The extraction is carried out twice at reflux for one hour.

Filtration in vacuo is then carried out. The combined filtrates are concentrated to 2 volumes.

Three liquid-liquid extractions are carried out by addition of one volume of 0.1 M sodium carbonate (Na₂CO₃). The exhausted dichloromethane phase is retained. The residue obtained after drying over sodium sulphate, concentration and evaporation to dryness in vacuo at 60° C. constitutes “the macrocarpal-depleted extract” (the fraction by weight of macrocarpal G being less than 0.1%).

The combined basic aqueous phases are acidified by addition of 1 M hydrochloric acid (HCl) until a pH of approximately 1 is obtained, and then they are extracted by three liquid-liquid extractions with dichloromethane. The organic phases are dried over sodium sulphate and then concentrated and evaporated to dryness in vacuo at 60° C. maximum. The resulting dry residue constitutes “the extract enriched with macrocarpal G”. The latter contains a fraction by weight of macrocarpal G of 7%.

The enriched extract is fractionated on a preparative column having a reverse phase, Symetry Shield®, 5 μm (Waters), as the stationary phase and a mixture of acetonitrile/water/trifluoroacetic acid in the proportions 95/5/0.1% as the mobile phase.

The macrocarpal G purity of the resulting fraction is approximately 97%.

The protocol subsequently used is identical with that of Example 5 as regards the evaluation of serotonin reuptake. The results obtained are recorded in Table 2 below.

TABLE 2
Comparison of the activity of an “extract enriched with macrocarpal
G”, an “extract without macrocarpal G” and a fraction
enriched with macrocarpal G on serotonin reuptake

| | Concentrations | |
|---|---|---|
| | (μg of extract/ | % |
| Test | ml of solution) | inhibition |
| Extract enriched with macrocarpal G | 15 | 101 |
| Extract depleted of macrocarpal G | 15 | 37 |
| Macrocarpal G | 0.3 | 103.8 |

It is noted that the higher the macrocarpal G content, the more significant the percentage inhibition of serotonin reuptake.

### EXAMPLE 7

### Determination of the 50% Inhibitory Concentration (IC₅₀) of Macrocarpal A, Macrocarpal B, Macrocarpal C and Macrocarpal G on the Reuptake of the Neuromediators Compared with that of Hyperforin

The protocols followed are those of Example 5. They were repeated for different concentrations of macrocarpal A, B, C and G and of hyperforin.

The inhibition curves obtained enabled the following IC₅₀ values to be obtained:

TABLE 3
Determination of the 50% inhibitory concentration
(IC₅₀) of macrocarpal A, macrocarpal B, macrocarpal C and
macrocarpal G and of hyperforin on serotonin, noradrenaline
and dopamine reuptake

| | IC₅₀ (μg of extract/ml of solution) | | | | |
|---|---|---|---|---|---|
| Test | Macrocarpal A | Macrocarpal B | Macrocarpal C | Macrocarpal G | Hyperforin |
| Serotonin | 1.3 | 2.5 | 1.3 | 1.4 | 0.89 |
| reuptake | | | | | |
| Noradrenaline | 1.8 | >3.1 | 0.54 | 3.1 | 0.79 |
| reuptake | | | | | |
| Dopamine | 0.71 | 1.5 | 0.68 | 0.35 | 0.23 |
| reuptake | | | | | |

These results showed that the four compounds are carriers of neuromediator reuptake inhibiting activity. Moreover, the levels of activity of the four compounds are of equivalent order.

1-26. (canceled)

27. A method for the treatment and/or prevention of neurological or psychiatric diseases or pathologies and related disorders, of functional somatic syndromes and of dependence on addictive substances, arising from a disorder of dopamine and/or serotonin and/or noradrenaline reuptake comprising administering an effective amount of an Eucalyptus extract.

28. The method of claim 27, wherein dopamine and/or serotonin and/or noradrenaline reuptake is inhibited.

29. The method of claim 27, wherein the Eucalyptus extract comprises at least one compound selected from those of formula (I): 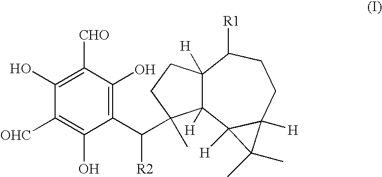
in which R1, together with the carbon atom to which it is bonded, forms a C═CH2 group or a group 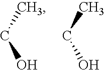
or 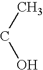
and R2 represents an isobutyl, α-isobutyl or β-isobutyl group; and diastereoisomeric forms thereof.

30. The method of claim 29, wherein the compound of formula (I) is macrocarpal A (5-((1R)-1-((11S,7R)-7-hydroxy-3,3,7,11-tetramethyltricyclo(6.3.0.0(2,4))undec-11-yl)-3-methylbutyl)-2,4,6-tri-hydroxybenzene-1,3-dicarbaldehyde) in which R1, together with the carbon atom to which it is bonded, forms the group 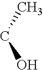
and R2 represents a β-isobutyl group; and wherein the weight percent thereof in the Eucalyptus extract is greater than or equal to 0.1% and less than 3%.

31. The method of claim 29, wherein the compound of formula (I) is macrocarpal B (5-((1S)-1-((11S,7R)-7-hydroxy-3,3,7,11-tetramethyltricyclo(6.3.0.0(2,4))undec-11-yl)-3-methylbutyl)-2,4,6-tri-hydroxybenzene-1,3-dicarbaldehyde) in which R1, together with the carbon atom to which it is bonded, forms the group 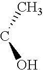
and R2 represents an α-isobutyl group; and wherein the weight percent thereof in the Eucalyptus extract is greater than or equal to 0. 1% and less than 3%.

32. The method of claim 29, wherein the compound of formula (I) is macrocarpal C (5-((1R)-1-((11S)-3,3,11-trimethyl-7-methylenetricyclo(6.3.0.0(2,4))undec-11-yl)-3-methylbutyl)-2,4,6-trihydroxybenzene-1,3-dicarbaldehyde) in which R1, together with the carbon atom to which it is bonded, forms the C═CH2 group and R2 represent a β-isobutyl group; and wherein the weight percent thereof in the Eucalyptus extract is greater than or equal to 0. 1% and less than 3%.

33. The method of claim 29, wherein the compound of formula (I) is macrocarpal G (5-(1-(3,3,11-trimethyl-7-methylenetricyclo-(6.3.0.0(2,4))undec-11-yl)-3-methylbutyl)-2,4,6-trihydroxybenzene-1,3-dicarbaldehyde) in which R1, together with the carbon atom to which it is bonded, forms a C═CH2 group and R2 represents an isobutyl group; and wherein the weight percent thereof in the Eucalyptus extract is greater than or equal to 0.1% and less than 5%.

34. The method of claim 29, wherein the Eucalyptus extract is enriched with at least one compound of formula (I).

35. The method of claim 34, wherein the Eucalyptus extract is enriched with macrocarpal A, and wherein the weight percent of macrocarpal A is greater than or equal to 3% and less than 90%.

36. The method of claim 34, wherein the Eucalyptus extract is enriched with macrocarpal B, and wherein the weight percent of macrocarpal B is greater than or equal to 3% and less than 90%.

37. The method of claim 34, wherein the Eucalyptus extract is enriched with macrocarpal C, and wherein the weight percent of macrocarpal C is greater than or equal to 3% and less than 90%.

38. The method of claim 34, wherein the Eucalyptus extract is enriched with macrocarpal G, and wherein the weight of macrocarpal G is greater than or equal to 5% and less than 90%.

39. The method of claim 27, wherein the extract is obtained from a Eucalyptus selected from the species belonging to the subgenera Eudesmia, Symphomyrtus and Corymbia and the following species: Eucalyptus globulus L., Eucalyptus pulverulenta Sims, Eucalyptus kartzoffiana L. A. S. Johnson 1 Blaxell, Eucalyptus macrocarpa Hook., Eucalyptus cinerea F. Muell.ex Benth., Eucalyptus dorrigoensis (Blakely) L. A. S. Johnson 1 K. D. Hill, Eucalyptus leptopoda Benth., Eucalyptus occidentalis Endl., Eucalyptus viridis R. T. Baker, Eucalyptus polybractea R. T. Baker and Eucalyptus smithii R. T. Baker.

40. The method of claim 27, wherein the Eucalyptus extract is selected from an extract of Eucalyptus leaves, flowers, fruits, stems and trunk.

41. The method of claim 27, wherein the neurological or psychiatric pathology or disease or related disorder, the functional somatic syndrome or the dependence on addictive substances is selected from neurological diseases, neurodegenerative diseases, amyotrophic lateral sclerosis, senile dementia, fronto-temporal dementia, vascular dementia, migraine, neuropathic pain of central origin, psychiatric diseases, depression, breakdown, schizophrenia, bipolar syndrome, general anxiety, stress-related diseases, panic attacks, obsessive compulsive disorders, post-traumatic stress syndromes, attention and hyperactivity disorders, eating disorders, phobia, autism, functional somatic syndromes, and memory, attention and vigilance disorders related to neurological pathologies or psychiatric disorders.

42. The method of claim 41, wherein the neurodegenerative disease is selected from Alzheimer's disease, Huntington's chorea, Parkinson's disease, cerebral vascular accidents and cranial traumatism.

43. The method of claim 41, wherein the depression is selected from endogenous, resistant, reactive and iatrogenic depression.

44. The method of claim 41, wherein the eating disorder is selected from bulimia and anorexia.

45. The method of claim 41, wherein the phobia is agoraphobia.

46. The method of claim 41, wherein the addictive substance is selected from nicotine, alcohol, opiates, cannabinoids, and psycho-stimulants.

47. The method of claim 41, wherein the functional somatic syndrome is selected from chronic fatigue syndrome, fibromyalgia, irritable bowel syndrome, gastro-oesophageal reflux, loss of libido, erectile dysfunction and urinary incontinence.

48. The method of claim 27, wherein the Eucalyptus extract is in the form of a pharmaceutical product or a food supplement.

49. The method of claim 48, wherein the pharmaceutical product is in oral or injectable form.

50. A method for the treatment and/or prevention of neurological or psychiatric diseases or pathologies and related disorders, of functional somatic syndromes and of dependence on addictive substances, arising from a disorder of dopamine and/or serotonin and/or noradrenaline reuptake comprising administering an effective amount of a compound selected from those of formula (I): 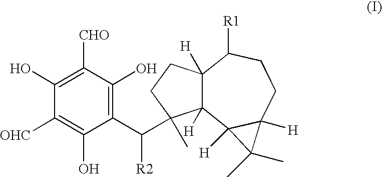
in which R1, together with the carbon atom to which it is bonded, forms a C═CH2 group or a group 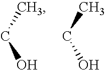
or 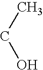
and R2 represents an isobutyl, α-isobutyl or β-isobutyl group; and diastereoisomeric forms thereof.

51. The method of claim 50, wherein dopamine and/or serotonin and/or noradrenaline reuptake is inhibited.

52. The method of claim 50, wherein the compound of formula (I), is:
macrocarpal A (5-((1R)-1-((11S,7R)-7-hydroxy-3,3,7,11-tetramethyltricyclo(6.3.0.0(2,4))undec-11-yl)-3-methylbutyl)-2,4,6-tri-hydroxybenzene-1,3-dicarbaldehyde) in which R1, together with the carbon atom to which it is bonded, forms the group 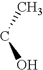
and R2 represents a β-isobutyl group;
macrocarpal B (5-((1S)-1-((11S,7R)-7-hydroxy-3,3,7,11-tetramethyltricyclo(6.3.0.0(2,4))undec-11-yl)-3-methylbutyl)-2,4,6-trihydroxybenzene-1,3-dicarbaldehyde) in which R1, together with the carbon atom to which it is bonded, forms the group 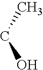
and R2 represents an α-isobutyl group;
macrocarpal C (5-((1R)-1-((11S)-3,3,11-trimethyl-7-methylenetricyclo-(6.3.0.0(2,4))undec-11-yl)-3-methylbutyl)-2,4,6-trihydroxybenzene-1,3-dicarbaldehyde) in which R1, together with the carbon atom to which it is bonded, forms the C═CH2 group and R2 represents a β-isobutyl group;
macrocarpal G (5-(1-(3,3,11-trimethyl-7-methylenetricyclo(6.3.0.0(2,4))-undec-11-yl)-3-methylbutyl)-2,4,6-trihydroxybenzene-1,3-dicarbaldehyde) in which R1, together with the carbon atom to which it is bonded, forms a C═CH2 group and R2 represents an isobutyl group; and their respective diastereoisomeric forms.

53. The method of claim 52, wherein the macrocarpal A, macrocarpal B, macrocarpal C or macrocarpal G is obtained by chemical or biochemical synthesis or from a plant extract.

54. The method of claim 50, wherein the neurological or psychiatric pathology or disease or related disorder, the functional somatic syndrome or the dependence on addictive substances is selected from neurological diseases, neurodegenerative diseases, amyotrophic lateral sclerosis, senile dementia, fronto-temporal dementia, vascular dementia, migraine, neuropathic pain of central origin, psychiatric diseases, depression, breakdown, schizophrenia, bipolar syndrome, general anxiety, stress-related diseases, panic attacks, obsessive compulsive disorders, post-traumatic stress syndromes, attention and hyperactivity disorders, eating disorders, phobia, autism, functional somatic syndromes, and memory, attention and vigilance disorders related to neurological pathologies or psychiatric disorders.

55. The method of claim 54, wherein the neurodegenerative disease is selected from Alzheimer's disease, Huntington's chorea, Parkinson's disease, cerebral vascular accidents and cranial traumatism.

56. The method of claim 54, wherein the depression is selected from endogenous, resistant, reactive and iatrogenic depression.

57. The method of claim 54, wherein the eating disorder is selected from bulimia and anorexia.

58. The method of claim 54, wherein the phobia is agoraphobia.

59. The method of claim 54, wherein the addictive substance is selected from nicotine, alcohol, opiates, cannabinoids, and psycho-stimulants.

60. The method of claim 54, wherein the functional somatic syndrome is selected from chronic fatigue syndrome, fibromyalgia, irritable bowel syndrome, gastro-oesophageal reflux, loss of libido, erectile dysfunction and urinary incontinence.

61. The method of claim 50, wherein the compound of formula (I), or a diastereoisomeric form thereof, is in the form of a pharmaceutical product or a food supplement.

62. The method of claim 61, wherein the pharmaceutical product is in oral or injectable form.

63. An Eucalyptus extract, comprising at least one compound selected from those of formula (I): 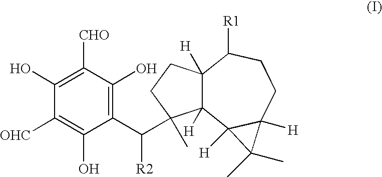
in which R1, together with the carbon atom to which it is bonded, forms a C═CH2 group or a group 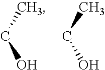
or 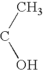
and R2 represents an isobutyl, α-isobutyl or β-isobutyl group; and diastereoisomeric forms thereof; and wherein:
the weight percent of macrocarpal A (5-((1R)-1-((11S,7R)-7-hydroxy-3,3,7,11-tetramethyltricyclo(6.3.0.0(2,4))undec-11-yl)-3-methylbutyl)-2,4,6-trihydroxybenzene-1,3-dicarbaldehyde) in which R1, together with the carbon atom to which it is bonded, forms the group 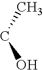
and R2 represents a β-isobutyl group, is greater than or equal to 3% and less than 90%;
the weight percent of macrocarpal B (5-((1S)-1-((11S,7R)-7-hydroxy-3,3,7,11-tetramethyltricyclo(6.3.0.0(2,4))undec-11-yl)-3-methylbutyl)-2,4,6-trihydroxybenzene-1,3-dicarbaldehyde) in which R1, together with the carbon atom to which it is bonded, forms the group 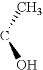
and R2 represents an α-isobutyl group, is greater than or equal to 3% and less than 90%;
the weight percent of macrocarpal C (5-((1R)-1-((11S)-3,3,11-trimethyl-7-methylenetricyclo(6.3.0.0(2,4))undec-11-yl)-3-methylbutyl)-2,4,6-trihydroxybenzene-1,3-dicarbaldehyde) in which R1, together with the carbon atom to which it is bonded, forms the C═CH2 group and R2 represents a β-isobutyl group, is greater than or equal to 3% and less than 90%; and
the weight percent of macrocarpal G (5-(1-(3,3,11-trimethyl-7-methylenetricyclo(6.3.0.0(2,4))undec-11-yl)-3-methylbutyl)-2,4,6-trihydroxybenzene-1,3-dicarbaldehyde) in which R1, together with the carbon atom to which it is bonded, forms a C═CH2 group and R2 represents an isobutyl group, is greater than or equal to 5% and less than 90%.

64. A process for the preparation of the extract of claim 63, comprising the following steps: grinding Eucalyptus leaves and/or flowers and/or fruits and/or stems and/or trunk; extracting at least once with supercritical fluid with or without a co-solvent; recovering the extract and optionally drying the extract, or extracting at least once with an organic solvent or a mixture of water and water-miscible organic solvents, solid/liquid separation; and enriching the filtrate.

65. The process of claim 64, wherein the organic solvent is dichloromethane or isopropyl acetate.

66. The process of claim 64, wherein enriching the filtrate comprises at least one liquid/liquid extraction by addition of a base, acidification and then at least one liquid/liquid extraction using a water-immiscible solvent.
